(19) United States
(12) Patent Application Publication
(10) Pub. No.: US 2020/0209043 A1
(43) Pub. Date: Jul. 2, 2020

(54) CAPACITIVE SENSING FOR PRIMING OF DIALYSIS MACHINES

(71) Applicant: Fresenius Medical Care Holdings, Inc., Waltham, MA (US)

(72) Inventors: Hernando G. Garza, Concord, CA (US); Asif A. Khan, Hercules, CA (US); Philip S. James, Orinda, CA (US); Micah S. Langley, Oakley, CA (US); Benjamin W. Allen, Novato, CA (US); Kulwinder S. Plahey, Martinez, CA (US)

(21) Appl. No.: 16/233,593

(22) Filed: Dec. 27, 2018

(51) Int. Cl. G01F 23/26; A61M 1/28; G08B 21/18

(52) U.S. Cl. CPC G01F 23/266; A61M 39/10; G08B 21/182; A61M 1/288

(57) ABSTRACT

A dialysis system may include a dialysis machine (e.g., a peritoneal dialysis machine) having a housing. Tubing may be extendable between the dialysis machine and a patient for fluid delivery from a container to the patient during a dialysis treatment. A connector may be attachable to the housing and configured to receive at least an end of the tubing. A capacitive sensor may be disposed in proximity to the connector. In connection with priming the tubing, prior to the dialysis treatment, a fluid may be flowable through the tubing from the container to the end of the tubing at the connector such that a presence of the fluid at the end of the tubing may be detectable by the capacitive sensor.

### FIELD

The disclosure generally relates to a dialysis system, and more particularly to a detector in a dialysis machine and methods for detecting a priming of tubing prior to a dialysis treatment.

### BACKGROUND

Dialysis machines are known for use in the treatment of renal disease. The two principal dialysis methods are hemodialysis (HD) and peritoneal dialysis (PD). During hemodialysis, the patient's blood is passed through a dialyzer of a hemodialysis machine while also passing dialysate through the dialyzer. A semi-permeable membrane in the dialyzer separates the blood from the dialysate within the dialyzer and allows diffusion and osmosis exchanges to take place between the dialysate and the blood stream. During peritoneal dialysis, the patient's peritoneal cavity is periodically infused with dialysate or dialysis solution. The membranous lining of the patient's peritoneum acts as a natural semi-permeable membrane that allows diffusion and osmosis exchanges to take place between the solution and the blood stream. Automated peritoneal dialysis machines, called PD cyclers, are designed to control the entire peritoneal dialysis process so that it can be performed at home, usually overnight, without clinical staff in attendance.

A dialysis machine, such as a peritoneal dialysis machine, may include one or more containers (e.g., bags) containing a fluid, e.g., a dialysate, for patient infusion. In peritoneal dialysis machines, for example, tubing as one or more fluid lines are inserted into an abdomen of a patient for flowing fresh dialysate and removing used dialysate, waste, and excess fluid. Prior to patient insertion and a dialysis treatment, the tubing is primed with dialysate to minimize air in the tubing being delivered to the peritoneal cavity of the patient, which may cause cramps or discomfort.

It is with respect to these and other considerations that the present improvements may be useful.

### SUMMARY

This Summary is provided to introduce a selection of concepts in a simplified form that are further described below in the Detailed Description. This Summary is not intended to necessarily identify key features or essential features of the claimed subject matter, nor is it intended as an aid in determining the scope of the claimed subject matter.

According to an exemplary embodiment of the present disclosure, a dialysis machine may include a housing. A connector may be attachable to the housing, and the connector may be configured to receive at least an end of tubing. A capacitive sensor may be disposed in proximity to the connector. A fluid may be flowable through the tubing to the end of the tubing at the connector, such that a presence of the fluid at the end of the tubing may be detectable by the capacitive sensor.

According to an exemplary embodiment of the present disclosure, a method for priming tubing of a dialysis machine may include attaching an end of the tubing to a connector coupled to a housing of the dialysis machine. Fluid may be delivered from a first container through the tubing to the connector. A presence of the fluid may be detected at the end of the tubing by a capacitive sensor disposed in proximity to the connector.

According to an exemplary embodiment of the present disclosure, a dialysis system may include a dialysis machine including a housing. Tubing may be extendable between the dialysis machine and a patient for fluid delivery from a container to the patient during a dialysis treatment. A connector may be attachable to the housing and may be configured to receive at least an end of the tubing. A capacitive sensor may be disposed in proximity to the connector. Prior to the dialysis treatment, a fluid may be flowable through the tubing from the container to the end of the tubing at the connector such that a presence of the fluid at the end of the tubing may be detectable by the capacitive sensor.

In various of the foregoing and other embodiments of the present disclosure, the connector may include a hydrophobic filter. In response to the capacitive sensor detecting the presence of the fluid, an alert may be generated to perform a visual verification of the tubing. In response to a lack of presence of the fluid in the visual verification of the tubing, an alarm may be generated. The alarm may be generated by the dialysis machine. The alarm may be generated by the dialysis system. In response to a confirmation of presence of the fluid in the visual verification of the tubing, a dialysis treatment may be performed. A timer may be included for timing the fluid flow through the tubing to the connector. In response to exceeding a predetermined time to detect the presence of the fluid, an alarm may be generated. The alarm may be generated by the dialysis machine. The alarm may be generated by the dialysis system. The capacitive sensor may be disposed in the dialysis machine in proximity to the connector, such that a capacitance in the sensor may switch in response to detecting the presence of the fluid within a predetermined distance to the sensor. The dialysis machine may be a peritoneal dialysis machine.

### BRIEF DESCRIPTION OF THE DRAWINGS

By way of example, specific embodiments of the disclosed machine will now be described, with reference to the accompanying drawings, in which:

FIG. 1 illustrates an exemplary embodiment of a dialysis machine in accordance with the present disclosure;

FIG. 2 illustrates an exemplary embodiment of a connector for a dialysis machine in accordance with the present disclosure;

FIGS. 3A-3B illustrate an exemplary embodiment of a dialysis system in accordance with the present disclosure;

FIG. 4 illustrates an exemplary embodiment of a dialysis machine in accordance with the present disclosure;

FIG. 5 illustrates an exemplary embodiment of a method for operating a dialysis machine in accordance with the present disclosure; and

FIG. 6 illustrates exemplary embodiments of an electrical circuit of a dialysis machine in accordance with the present disclosure.

### DETAILED DESCRIPTION

The present embodiments will now be described more fully hereinafter with reference to the accompanying drawings, in which several exemplary embodiments are shown. The subject matter of the present disclosure, however, may be embodied in many different forms and should not be construed as limited to the embodiments set forth herein. Rather, these embodiments are provided so that this disclosure will be thorough and complete, and willfully convey the scope of the subject matter to those skilled in the art. In the drawings, like numbers refer to like elements throughout.

As described above, in peritoneal dialysis operations, tubing is connected between a dialysis machine and a patient for delivering fresh dialysate into the patient's peritoneal cavity, and removing used dialysate and contaminants after a predetermined time. A patient may undergo several cycles of delivering a fresh batch of dialysate and removing the used dialysate and contaminants in a single treatment. In some embodiments, a peritoneal dialysis treatment may be performed at home, and may occur overnight while a patient is sleeping.

To minimize discomfort so that a patient may be able to sleep through the treatment, tubing extending between the dialysis machine and the patient may be primed with dialysate prior to the dialysis treatment. Tubing is primed when a fluid (e.g., dialysate) is flowed through the tubing prior to being inserted in the patient to minimize, or eliminate, any air pockets present in the tubing. Priming minimizes or prevents air infusion to the peritoneal cavity of the patient, thereby minimizing potential pain, cramps, and/or other discomfort during the dialysis treatment. However, verification of full priming of the tubing may be challenging. A patient may have difficulty manually checking the full length of the tubing as the fluid (e.g., dialysate) may be clear, the tubing may be several feet long, and/or several additional steps must be performed to set up the dialysis machine for treatment. Semi- or fully-automated checks may generally include a timer in the dialysis machine, so that fluid is flowed through the tubing for a predetermined time period, which may align with a length of the tubing. Additionally, pressure sensors and/or programmed volume verifications may be included, although these may require direct contact with the fluid.

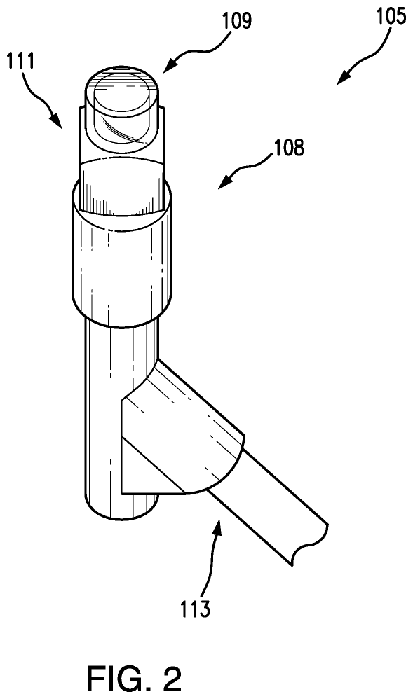

Exemplary embodiments of the present disclosure for a priming detector for dialysis machines may include a capacitive sensor for non-fluid contact to detect a presence of fluid at an end of the tubing. The tubing may be positioned such that an end of the tubing is within a predetermined proximity to the capacitive sensor. Fluid (e.g., dialysate) may be flowed through the tubing from a first end coupled to a container (e.g., a dialysate bag) to a second end. In some embodiments, the second end of the tubing may be attached to a connector (see FIG. 2). When the fluid reaches the second end of the tubing, the capacitive sensor may sense a change in capacitance resulting from the presence of the fluid in the predetermined proximity to the sensor and switch, thereby alerting a user that the fluid has flowed through the length of the tubing.

In embodiments, dialysate may be stored in containers, e.g., a flexible bag, that may be formed of a Biofine™ material and/or a polyvinyl chloride (PVC) material. Although the term “bag” is used throughout, it should be understood that a dialysate bag may be any type of container capable of holding a fluid, e.g., a dialysate. In some embodiments, a fluid container may include a container in which dry concentrates are mixed with water to generate dialysate suitable for a dialysis treatment.

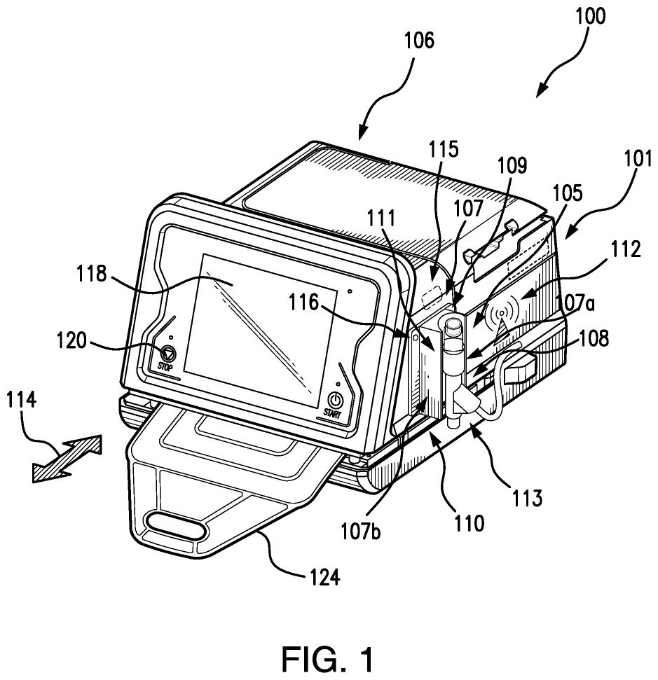

Referring now to FIG. 1, an exemplary embodiment of a dialysis machine 100 in accordance with the present disclosure is shown. It is understood that the dialysis machine 100 may be implemented in a peritoneal dialysis system, and may include, for example, a housing 106, a processing module 101, a connectivity component 112, a touch screen 118, and a control panel 120 operable by a user (e.g., a caregiver or a patient) to allow, for example, set up, initiation, and/or termination of a dialysis treatment. The processing module 101 may be configured to receive data from the touch screen 118, the control panel 120 and sensors, e.g., weight, air, flow, temperature, and/or pressure sensors, and control the dialysis machine 100 based on the received data. For example, the processing module 101 may adjust the operating parameters of the dialysis machine 100. The connectivity component 112 may be a transceiver for wireless connections and/or other signal processor for processing signals transmitted and received over a wired connection. Other medical devices (e.g., other dialysis machines) or components may be configured to connect to the network 311 and communicate with the dialysis machine 100.

One or more heating elements may be disposed internal to the dialysis machine 100. For example, a warmer pouch 124 may be insertable into an opening 110 in a direction indicated at arrow 114. It is also understood that the warmer pouch 124 may be connectable to the dialysis machine 100 via tubing, or fluid lines, via a cartridge. The tubing may be connectable so that dialysate may flow from containers (e.g., dialysate bags), through the warmer pouch 124 for heating, and to the patient.

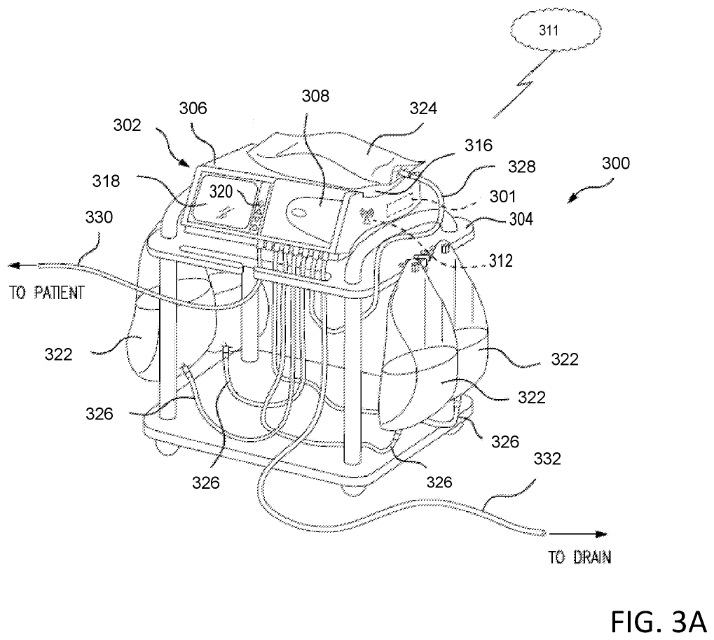

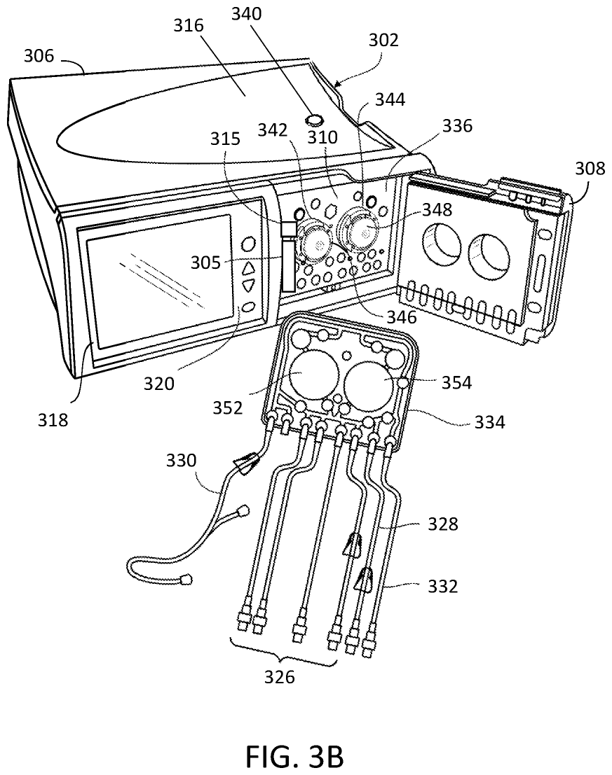

In such in-line heating embodiments, the warmer pouch 124 may be configured so dialysate may continually flow through the warmer pouch to achieve a predetermined temperature before flowing into the patient. Internal heating elements (not shown) may be positioned above and/or below the opening 110, so that when the warmer pouch 124 is inserted into the opening 110, the one or more heating elements may affect the temperature of dialysate flowing through the warmer pouch 124. In some embodiments, the internal warmer pouch may instead be a portion of tubing in the system that is passed by, around, or otherwise configured with respect to, a heating element(s). It is understood that FIG. 1 illustrates dialysate continuously flowing through the warmer pouch 124 “in-line” with the dialysis machine 100, reaching an acceptable temperature by the application of internal heating elements, and that FIGS. 3A-3B, as described below, illustrate that dialysate may be transferable to and stored in the heater bag 324 by “batch” until reaching an acceptable temperature for use.

In embodiments, a patient line may be connected to the cartridge. The patient line may be connectable to a patient's abdomen (e.g., peritoneal cavity) via a catheter and may be used to pass dialysate back and forth between the cartridge and the patient's peritoneal cavity by the pump heads during use.

As described above, prior to insertion of the patient line into the patient, the tubing may be primed with fluid (e.g., dialysate), to purge air from the tubing. As shown in FIGS. 1 and 2, a patient line connector 105 may be attachable to the housing 106 of the dialysis machine 100. In some embodiments, the patient line connector 105 may have a substantially cylindrical body 108. The cylindrical body 108 may be at least partially formed of a transparent or translucent material, to aid in a manual visual verification of a presence of fluid. A filter 109, e.g., a hydrophobic filter, may be disposed at an end of the cylindrical body 108. The filter 109 may have a membrane for air to escape as the tubing is primed, e.g., fluid is flowed through the tubing from the container in a direction towards the connector 105.

The connector 105 may be detachably coupleable to the housing 106 of the dialysis machine 100 by a bracket 107. The bracket 107 may be configured to receive at least a portion of the patient line connector 105. The connector 105 may be attachable to the housing 106 via the bracket 107 such that the filter 109 is vertically upright, e.g., at a top portion 111 of the connector 105. In this manner, fluid may be prevented from flowing out of the filter 109 while allowing air to escape during a priming operation.

In some embodiments, the bracket 107 may include one or more protrusions 107a, 107b, which may be configured as a press fit, or an interference fit, to retain the connector 105 in the desired vertical alignment. It is also understood that the bracket 107 may be any configuration to attach to the housing 106 of the dialysis machine and to receive and retain the connector 105, including but not limited to clips, snaps, and the like. In embodiments, the bracket 107 may be a separate component attachable to the housing 106, although it is also envisioned that the bracket may be integrally formed in the housing 106, e.g., as a molded insert.

In embodiments, the connector 105 may be configured to receive at least an end of the patient line, or tubing, 113. The end 113 may be attachable into the connector in a known manner, such that as a length of the tubing is primed with fluid (e.g., dialysate), air in the line may be purged, or pushed to the end 113 and may escape via the filter 109. When the fluid is flowed to the end 113, at least a portion of the fluid may enter the connector 105. Upon reaching the connector 105, a presence of fluid may be detectable by one or more sensors.

As described above, a capacitive sensor 115 may be disposed within the housing 106. In some embodiments, a capacitive sensor 115 may be disposed outside of the housing 106, e.g., not in fluid contact with the connector 105, and/or attachable directly to the tubing. The capacitive sensor 115 may be advantageous over other known sensors for detecting patient line priming in that no contact with the fluid is necessary for the capacitance to switch and alert a user when a presence of fluid is detected. Additionally, in some embodiments the capacitive sensor 115 may be utilized in a dialysis system to monitor or detect a desired fluid level. The capacitive sensor 115 may be configured in a known manner, e.g., such as a sensor provided by Omron Electronics LLC, or Gems Sensors & Controls, and may include one or more electrodes embedded in the sensor 115 to detect changes in capacitance as fluid is within a predetermined distance from a face of the sensor 115. The capacitive sensor 115 may be capable of sensing a presence of fluid, or no fluid, at the predetermined distance, in the tubing. At a pre-set trigger point, the capacitive sensor 115 may switch, and in some embodiments may alert a user to a presence of fluid. It is understood that the trigger point may be pre-calibrated prior to use, e.g., at the manufacturer, and/or the machine 100, 302 may self-calibrate the trigger point during a start-up procedure prior to performing a treatment.

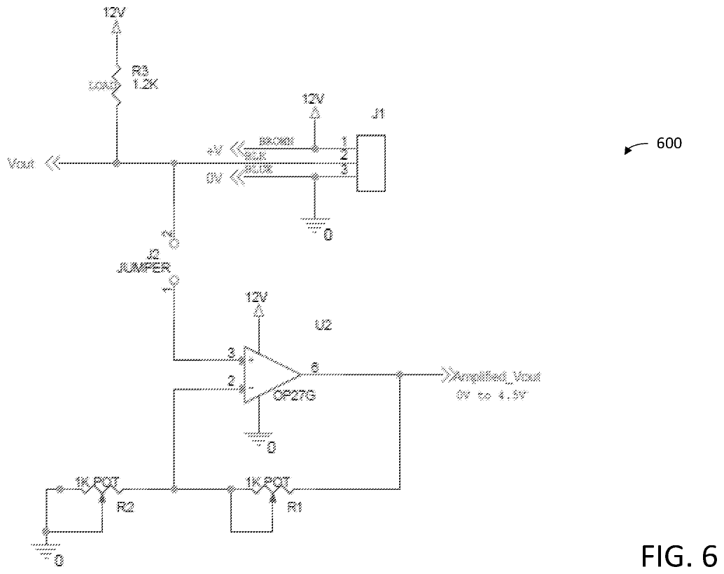

Referring now to FIG. 6, an exemplary embodiment of circuitry 600 for the capacitive sensor 115 is provided for illustrative purposes only, and other configurations may be envisioned, including but not limited to an operational amplifier or buffer for the output signal. In embodiments, the capacitive sensor 115 may be operatively connected to a logic board, or I/O board, and/or a processor of the dialysis machine 100, 302, 400. When the capacitance of the capacitive sensor 115 switches, e.g., a presence of fluid is detected within a predetermined distance, the dialysis machine 100, 302, 400, and/or the dialysis system 300, may alert a user. In some embodiments, an alert may be visual and/or audible. A visual alert may include a light emitting diode (LED) 116, and may be operatively connected in the circuitry 600. When the capacitance switches, LED 116 may be turned on, or may change to a different color, to alert a user to a presence of fluid at the connector 105. This may indicate to a user that a priming operation may be complete. In addition or alternatively to the LED 116, a notification may be displayable on the touch screen 118 to alert a user. In some embodiments, an audible alarm, such as a chime or other noise, may be emittable by the dialysis machine 100, 302, 400, and/or dialysis system 300, to indicate to a user that priming may be complete.

Referring now to FIGS. 3A-3B, an example of a dialysis system 300 (e.g., a peritoneal dialysis (PD) system) that is configured in accordance with an exemplary embodiment of the system described herein is shown. In some implementations, the dialysis system 300 may be configured for use at a patient's home (e.g., a home PD system). The dialysis system 300 may include a dialysis machine 302 (e.g., a peritoneal dialysis machine 302, also referred to as a PD cycler) and in some embodiments the machine may be seated on a cart 304. Although the dialysis system 300 is described and illustrated in connection with the dialysis machine 302, in other embodiments, the dialysis machine 100 may be included in or used in connection with the dialysis system 300.

It is also understood that any combination of the following components described with respect to the dialysis machine 302 may be similarly incorporated in the dialysis machine 100. The dialysis machine 302 may include a housing 306, a door 308, and a cartridge interface 310 including piston assemblies 342, 344 coupled to pump heads 346, 348 for contacting a disposable cassette, or cartridge 334, where the cartridge 334 is located within a compartment 336 formed between the cartridge interface 310 and the closed door 308, and which align with pump chambers 352, 354 formed in the cartridge 334. Fluid lines, or tubing 326, 328, 332, may be coupled to the cartridge 334, and may further include valves for controlling fluid flow to and from fluid bags including fresh dialysate and warming fluid. In another embodiment, at least a portion of the fluid lines may be integral to the cartridge 334. Prior to operation, a user may open the door 308 to insert a fresh cartridge 334, and to remove the used cartridge 334 after operation.

The cartridge 334 may be placed in the compartment 336 of the machine 302 for operation. During operation, dialysate fluid may be flowed into a patient's abdomen via the cartridge 334, and spent dialysate, waste, and/or excess fluid may be removed from the patient's abdomen via the cartridge 334. The door 308 may be securely closed to the machine 302. Peritoneal dialysis for a patient may include a total treatment of approximately 10 to 30 liters of fluid, where approximately 2 liters of dialysate fluid are pumped into a patient's abdomen, held for a period of time, e.g., about an hour, and then pumped out of the patient. This may be repeated until the full treatment volume is achieved, and usually occurs overnight while a patient sleeps. The dialysis machine 302 may also include a user interface such as a touch screen 318 and control panel 320 operable by a user (e.g., a caregiver or a patient) to allow, for example, set up, initiation, and/or termination of a dialysis treatment. The touch screen 318 and the control panel 320 may allow an operator to input various treatment parameters to the dialysis machine 302 and to otherwise control the dialysis machine 302. In addition, the touch screen 318 may serve as a display. The touch screen 318 may function to provide information to the patient and the operator of the dialysis system 300. For example, the touch screen 318 may display information related to a dialysis treatment to be applied to the patient, including information related to a prescription.

Dialysate bags 322 may be suspended from hooks on the sides of the cart 304. Hanging the dialysate bags 322 may improve air management as air content may be disposed by gravity to a top portion of the dialysate bag 322. Although four dialysate bags 322 are illustrated in FIG. 3A, any number “n” of dialysate bags may be connectable to the dialysis machine 302 (e.g., 1 to 5 bags, or more), and reference made to first and second bags is not limiting to the total number of bags used in a dialysis system 300. For example, the dialysis machine may have dialysate bags 322a, . . . 322n connectable in the system 300. In some embodiments, connectors and tubing ports may connect the dialysate bags 322 and lines for transferring dialysate.

The dialysis machine 302 may include a processing module 301 that resides inside the dialysis machine 302, the processing module 301 being configured to communicate with the touch screen 318 and the control panel 320. The dialysis machine 302 may be configured to connect to a network 311. The connection to network 311 may be via a wired and/or wireless connection. The dialysis machine 302 may include a connectivity component 312 configured to facilitate the connection to the network 311. The processing module 301 and the connectivity component 312 may be configured similarly to the processing module 101 and connectivity component 112 described above.

In some embodiments, a heater tray 316 may be positioned on top of the housing 306. The heater tray 316 may be any size and shape to accommodate a bag of dialysate (e.g., a 5 L bag of dialysate) for batch heating. In some embodiments, the heater tray 316 may include a heating element 340, for heating the dialysate prior to delivery into the patient. A heater bag 324 may be positioned in the heater tray 316. Dialysate from the dialysate bags 322 may be transferred to the heater bag 324 in batches. For example, a batch of dialysate may be transferred from the dialysate bags 322 to the heater bag 324, where the dialysate is heated by the heating element 340. When the batch of dialysate has reached a predetermined temperature (e.g., approximately 98°-100° F., 37° C.), the dialysate may be flowed into the patient.

The dialysate bags 322 and the heater bag 324 may be connected to the cartridge 334 via dialysate bag lines or tubing 326 and a heater bag line or tubing 328, respectively. The dialysate bag lines or tubing 326 may be used to pass dialysate from dialysate bags 322 to the cartridge during use, and the heater bag line 328 may be used to pass dialysate back and forth between the cartridge and the heater bag 324 during use. A drain line 332 may be connected to the cartridge 334 The drain line 332 may be connected to a drain or drain receptacle and may be used to pass dialysate from the cartridge to the drain or drain receptacle during use.

A patient line 330, as described above, may be connected to the cartridge 334, and may be attachable to a connector 305 for detecting a presence of fluid in the patient line prior to a dialysis treatment. As described above with respect to FIGS. 1 and 2, the connector 305 may be attachable to at least an end of the patient line 330, and a capacitive sensor 315 may be disposed in the dialysis machine 302 to detect a presence of fluid at the end of the patient line in the connector. Additional features and descriptions related to the connector and the capacitive sensor of FIGS. 1 and 2 may also be included in the dialysis machine 302.

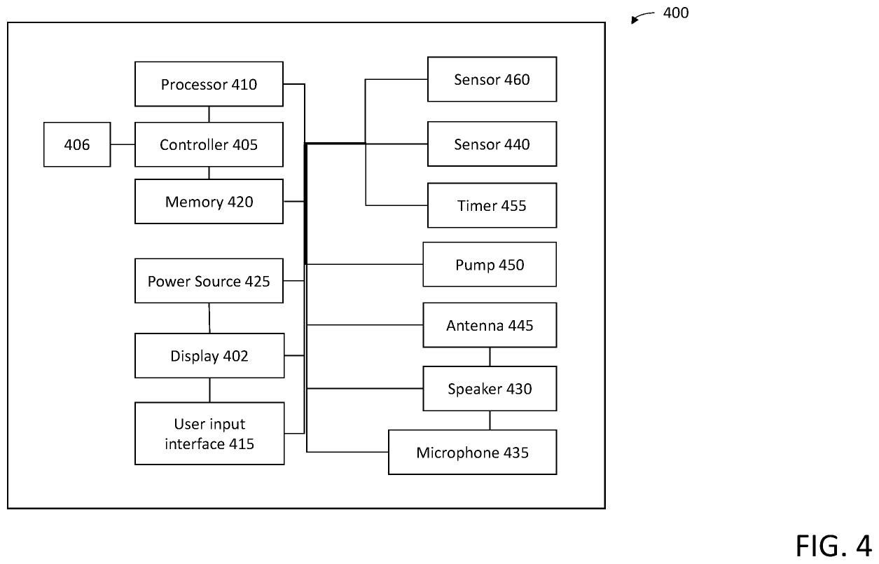

Referring to FIG. 4, a schematic of an exemplary embodiment of a dialysis machine 400 and a controller 405 in accordance with the present disclosure are shown. The dialysis machine 400 may be a home dialysis machine, e.g., a peritoneal dialysis machine, for performing a dialysis treatment on a patient, and may be included in the system 300 for dialysis machines 100, 302, described above. Additionally, components described with respect to the dialysis machine 400 may also be included in the dialysis machines 100, 302. It is understood that the dialysis machine 400 may be dialysis machines 100, 302, and/or may include any or all of the features of dialysis machines 100, 302. A power source 425 may provide power and/or a connection to an external power source to the dialysis machine 102, 302, 400.

The controller 405 may automatically control execution of a treatment function during a course of dialysis treatment. For example, the controller 405 may control the delivery and transfer of dialysate for dialysis machines 100, 302, 400. The controller 405 may be operatively connected to one or more sensors 440 and may deliver one or more signals to execute one or more treatment functions, or a course of treatment associated with various treatment systems. For example, dialysis treatment may include transferring dialysate from the dialysate bag 322 to the heater bag 324 and then to the patient, or delivering dialysate from the dialysate bag 322 through the warmer pouch 124 to the patient.

As described above, a sensor 460, e.g., a capacitive sensor 460, may be disposed in the dialysis machine 100, 302, 400, within a predetermined proximity to a bracket to receive a connector attached to an end of tubing, e.g., the patient line. In some embodiments, the capacitive sensor may be attachable to an outer surface of the dialysis machine, e.g., and may be detachably coupleable to a predetermined location in proximity to the connector. The capacitive sensor 460 may be configured to sense a presence of fluid in the connector and/or the end of the tubing during a priming operation, e.g., prior to treatment functions. In response to detecting a presence of fluid, the capacitance may switch, thereby triggering an alert to an user. The capacitive sensor 460 may be operatively connectable to an I/O board in communication with the processor 410, such that signals may be sent to a LED or other alerting function. As described above, the alert may be audible, such as a noise issued from a speaker 430, and/or visual, including an LED, a notification on the touch screen, or both.

In some embodiments, a timer 455 may be included for timing triggering of sensors 440, 460. It is understood that sensors, including but not limited to pressure sensors, weight sensors, flow sensors, air sensors, and temperature sensors, may detect dialysate temperature, fluid volume, fluid flow rate, and fluid flow pressure for the dialysis machine 100, 302, 400 to determine flow delivery to and from the patient. For example, the dialysis machine 100, 302, 400 may include a plurality of sensors for detection and/or measurement of any combination of temperature, pressure, volume, fluid flow. Multiple sensors may also be included to detect and/or measure individually the temperature, pressure, volume, fluid flow. Although FIG. 4 illustrates the components integral to the dialysis machine 400, at least one of the controller 405, processor 410, and/or memory 420 may be configured to be external and wired or wirelessly connected to the dialysis machine 100, 302, 400, as an individual component of a dialysis system. In some embodiments the controller 405, processor 410 and memory 420 may be remote to the dialysis machine and configured to communicate wirelessly.

According to exemplary embodiments of the present disclosure, the capacitive sensor 460 may be at least semi-independent of the timer 455. For example, fluid may be flowed through the patient line tubing from a container (e.g., dialysate bag) at a first end to a second end 113 including the connector 105 for any time period until the capacitive sensor 115, 460 detects a presence of fluid. In some embodiments, a timer 455 may be initiated when fluid begins to flow from the container through the tubing. The timer 455 may be used as a back-up, in the event the tubing has a kink, misconnection, leak, or other abnormality, or combinations thereof, such that the fluid does not flow to the end of the tubing. If the timer 455 expires prior to the capacitive sensor 115, 460 detecting the presence of fluid, an alarm may be generated to alert a user, to abort the priming operation, to pause the set-up and/or treatment operations, or combinations thereof.

In some embodiments, the controller 405, processor 410, and/or memory 420 of the dialysis machine 400 may receive sensor 440 signals indicating complete dialysate transfer of the dialysate bags, and indicating process parameters, such as temperature, pressure, volume, flow rate, and the like. The controller 405 may also detect connection of all dialysate bags 322 connected.

Communication between the controller 405 and the system may be bi-directional, whereby the system acknowledges control signals, and/or may provide state information associated with the system and/or requested operations. For example, system state information may include a state associated with specific operations to be executed by the system (e.g., trigger pump to deliver dialysate, trigger pumps and/or compressors to deliver filtered blood, and the like) and a status associated with specific operations (e.g., ready to execute, executing, completed, successfully completed, queued for execution, waiting for control signal, and the like).

In embodiments, the dialysis machine 100, 302, 400 may include at least one pump 450 operatively connected to the controller 405. During operation, the controller 405 may control the pump 450 for pumping fluid, e.g., fresh and spent dialysate, to and from a patient, and/or to prime the patient line tubing. The pump 450 may also pump dialysate from the dialysate bag 322 to the heater bag 324, or to another dialysate bag 322. In embodiments where the warmer pouch 124 is in-line with the dialysis machine 100, the pump 450 may pump the dialysate through the warmer pouch 124 directly to the patient during treatment. The controller 405 may also be operatively connected to a speaker 430 and a microphone 435 disposed in the dialysis machine 400, e.g., for generating audible alerts and/or alarms.

A user input interface 415 may include a combination of hardware and software components that allow the controller 405 to communicate with an external entity, such as a patient or other user, and a display 402 may display information to the user or medical professional. These components may be configured to receive information from actions such as physical movement or gestures and verbal intonation. In embodiments, the components of the user input interface 415 may provide information to external entities. Examples of the components that may be employed within the user input interface 415 include keypads, buttons, microphones, touch screens, gesture recognition devices, display screens, and speakers. The dialysis machine 100, 302, 400 may also be wirelessly connectable via the antenna 445 for remote communication.

As shown in FIG. 4, sensors 440 may be included for monitoring one or more treatment parameters, and sensor 460, e.g., capacitive sensor 460, for monitoring a priming operation, may be operatively connected to at least the controller 405, processor 410, and memory 420. Sensors 440 may include a pressure sensor for monitoring fluid pressure of the dialysis machine 100, 302, 400, although the sensors 440 may also include any of a heart rate sensor, a respiration sensor, a temperature sensor, a flow sensor, a weight sensor, a video sensor, an air sensor, an air bubble sensor, a thermal imaging sensor, an electroencephalogram sensor, a motion sensor, audio sensor, an accelerometer, or capacitance sensor. It is appreciated that the sensors 440, 460 may include sensors with varying sampling rates, including wireless sensors.

The processor 410 may be configured to execute an operating system, which may provide platform services to application software, e.g., for operating the dialysis machine 100, 302, 400. These platform services may include inter-process and network communication, file system management and standard database manipulation. One or more of many operating systems may be used, and examples are not limited to any particular operating system or operating system characteristic. In some examples, the processor 410 may be configured to execute a real-time operating system (RTOS), such as RT/Linux, or a non-real time operating system, such as BSD or GNU/Linux. As described above, it is also understood that the processor 410 may be operatively connected to an I/O board, for communication between the capacitive sensor 460 and a LED or other alerting function.

According to a variety of examples, the processor 410 may be a commercially available processor such as a processor manufactured by INTEL, AMD, MOTOROLA, and FREESCALE. However, the processor 410 may be any type of processor, multiprocessor or controller, whether commercially available or specially manufactured. For instance, according to one example, the processor 410 may include an MPC823 microprocessor manufactured by MOTOROLA.

The memory 420 may include a computer readable and writeable nonvolatile data storage medium configured to store non-transitory instructions and data. In addition, the memory 420 may include a processor memory that stores data during operation of the processor 410. In some examples, the processor memory includes a relatively high performance, volatile, random access memory such as dynamic random access memory (DRAM), static memory (SRAM), or synchronous DRAM. However, the processor memory may include any device for storing data, such as a non-volatile memory, with sufficient throughput and storage capacity to support the functions described herein. Further, examples are not limited to a particular memory, memory system, or data storage system.

The instructions stored on the memory 420 may include executable programs or other code that may be executed by the processor 410. The instructions may be persistently stored as encoded signals, and the instructions may cause the processor 410 to perform the functions described herein. The memory 420 may include information that is recorded, on or in, the medium, and this information may be processed by the processor 410 during execution of instructions. The memory 420 may also include, for example, specification of data records for user timing requirements, timing for priming or treatment and/or other operations, and historic sensor information. The medium may, for example, be optical disk, magnetic disk or flash memory, among others, and may be permanently affixed to, or removable from, the controller 405.

The controller 405 may be disposed in the dialysis machine 100, 302, 400 or may be coupled to the dialysis machine 100, 302, 400 via a communication port or wireless communication links, shown schematically as communication element 406 (see FIG. 3). According to various examples, the communication element 406 may support a variety of one or more standards and protocols, examples of which include USB, WiFi, TCP/IP, Ethernet, Bluetooth, Zigbee, CAN-bus, IP, IPV6, UDP, UTN, HTTP, HTTPS, FTP, SNMP, CDMA, NMEA and/or GSM. As a component disposed within the dialysis machine 400, the controller 405 may be operatively connected to any one or more of the sensors 440, 460, pump 450, or combinations thereof. The controller 405 may communicate control signals or triggering voltages to the components of the dialysis machine 100, 302, 400. As discussed, exemplary embodiments of the controller 405 may include wireless communication interfaces. The controller 405 may detect remote devices to determine if any remote sensors are available to augment any sensor data being used to evaluate the patient.

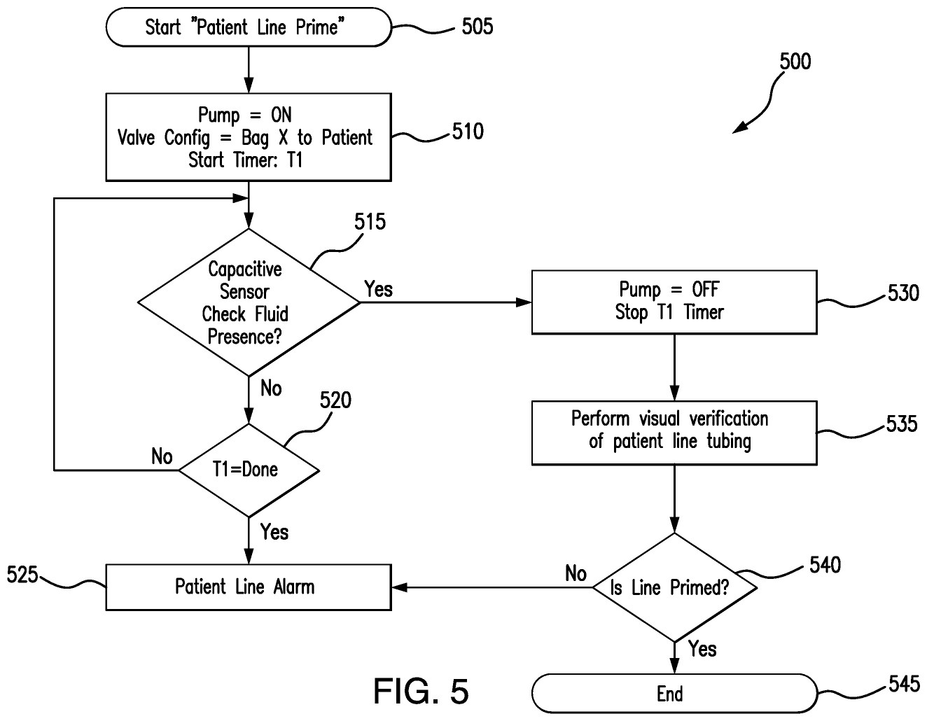

Referring now to FIG. 5, an exemplary flow diagram 500 of a method for priming tubing of a dialysis machine prior to a treatment operation is illustrated. It is understood that the dialysis machines 100, 302, 400 may be utilized in the exemplary method, and may be included in the system 300 as described above. At step 505, a user, e.g., a patient or medical caregiver, may being a priming operation. For example, the priming operation may occur prior to a dialysis treatment, and may be initiated via a touch screen or other user input to run a priming operation program in the dialysis machine. When the priming operation has been initiated, at step 510 a pump (e.g., piston assemblies 342, 344 coupled to pump heads 346, 348, 344, 450) may be turned on, so that fluid may pumped through the patient line tubing. For example, tubing may extend from a container (e.g., dialysate bag) at a first end to a second end, where the pumps may pump the fluid (e.g., dialysate) through the length of the tubing. In this manner, air may be purged or reduced from the tubing, by pushing the air to the second end of the tubing, which may be attached to a connector having a filter (e.g., hydrophobic filter). In some embodiments, at step 510, a timer (e.g., timer 455) may be started.

Once fluid is being pumped through the fluid lines, the capacitive sensor may be checked for a change in capacitance, or sensing a presence of fluid at step 515. If no fluid is detected, the timer may be checked at step 520 to determine if the predetermined time has been exceeded. For example, the capacitive sensor may check for a fluid presence for a predetermined period of time. If the predetermined time period has not been exceeded, the capacitive sensor may continue checking for a fluid presence and repeat to step 515. In response to exceeding a predetermined time period, an alarm may be generated at step 525. As described above, an alarm may alert a user to an unacceptable condition, such as kinked tubing, a leak, a misconnection, or the like, such that a treatment procedure may not occur. The alarm may pause or cancel the priming operation, so that the user may perform a manual check of the tubing and other equipment.

When the capacitive sensor detects a presence of the fluid within the predetermined time period, at step 530 the pump(s) may be turned off, and/or the timer may be cleared. In some embodiments, at step 535, a visual and/or audible alert may provide notification to the user or caregiver that a presence of fluid has been detected, e.g., the patient line may be primed. The alert may further indicate to the user or caregiver to perform a visual verification of the tubing. In this manner, a notification may be generated on the touch screen of the dialysis machine as a prompt to the patient or caregiver to perform this visual verification. The patient or caregiver may perform a manual check of the connector and/or end of the tubing connected to the housing of the dialysis machine for a presence of fluid at step 540. As described above, the connector may be formed of a substantially translucent and/or transparent material.

In response to confirming a presence of fluid at the end of the tubing and/or the connector in the visual verification step, the priming operation may end at step 545, and the patient may proceed to other set-up operations, and/or the treatment procedure. In response to a lack of presence of fluid at the end of the tubing and/or the connector in the visual verification step, a user or caregiver may enter the information in the dialysis machine, e.g., via a touch screen. An alarm may subsequently be generated, e.g., at step 525, to pause and/or cancel the priming operation. The patient may be able to determine if the fluid has not reached the connector and the end of the tubing, and if not, contributing factors. For example, if the capacitive sensor incorrectly or inadvertently detected a presence of fluid, the capacitive sensor may require recalibration or other maintenance or replacement. Another condition may include uncontained fluid in an area of the capacitive sensor, possibly generating a false-positive indicator.

Some embodiments of the disclosed system may be implemented, for example, using a storage medium, a computer-readable medium or an article of manufacture which may store an instruction or a set of instructions that, if executed by a machine (i.e., processor or microcontroller), may cause the machine to perform a method and/or operation in accordance with embodiments of the disclosure. In addition, a server or database server may include machine readable media configured to store machine executable program instructions. Such a machine may include, for example, any suitable processing platform, computing platform, computing device, processing device, computing system, processing system, computer, processor, or the like, and may be implemented using any suitable combination of hardware, software, firmware, or a combination thereof and utilized in systems, subsystems, components, or sub-components thereof. The computer-readable medium or article may include, for example, any suitable type of memory unit, memory device, memory article, memory medium, storage device, storage article, storage medium and/or storage unit, for example, memory (including non-transitory memory), removable or non-removable media, erasable or non-erasable media, writeable or re-writeable media, digital or analog media, hard disk, floppy disk, Compact Disk Read Only Memory (CD-ROM), Compact Disk Recordable (CD-R), Compact Disk Rewriteable (CD-RW), optical disk, magnetic media, magneto-optical media, removable memory cards or disks, various types of Digital Versatile Disk (DVD), a tape, a cassette, or the like. The instructions may include any suitable type of code, such as source code, compiled code, interpreted code, executable code, static code, dynamic code, encrypted code, and the like, implemented using any suitable high-level, low-level, object-oriented, visual, compiled and/or interpreted programming language.

As used herein, an element or operation recited in the singular and proceeded with the word “a” or “an” should be understood as not excluding plural elements or operations, unless such exclusion is explicitly recited. Furthermore, references to “one embodiment” of the present disclosure are not intended to be interpreted as excluding the existence of additional embodiments that also incorporate the recited features.

While the systems and techniques described herein for priming have been largely explained with reference to a dialysis machine, in particular, a peritoneal dialysis machine, the systems and techniques described for priming may be used in connection with other types of medical treatment systems and/or machines, such as a hemodialysis machine or other medical treatment device involving medical fluids. In some implementations, the dialysis machine may be configured for use in a patient's home (e.g., a home dialysis machine). The home dialysis machine can take the form of a peritoneal dialysis machine or a home hemodialysis machine.

The present disclosure is not to be limited in scope by the specific embodiments described herein. Indeed, other various embodiments of and modifications to the present disclosure, in addition to those described herein, will be apparent to those of ordinary skill in the art from the foregoing description and accompanying drawings. Thus, such other embodiments and modifications are intended to fall within the scope of the present disclosure. Furthermore, although the present disclosure has been described herein in the context of a particular implementation in a particular environment for a particular purpose, those of ordinary skill in the art will recognize that its usefulness is not limited thereto and that the present disclosure may be beneficially implemented in any number of environments for any number of purposes. Accordingly, the claims set forth below should be construed in view of the full breadth and spirit of the present disclosure as described herein.

What is claimed is:

1. A dialysis machine, comprising:
a housing;
a connector attachable to the housing and configured to receive at least an end of tubing;
a capacitive sensor disposed in proximity to the connector;
wherein a fluid is flowable through the tubing to the end of the tubing at the connector, such that a presence of the fluid at the end of the tubing is detectable by the capacitive sensor.

2. The dialysis machine according to claim 1, wherein the connector includes a hydrophobic filter.

3. The dialysis machine according to claim 1, wherein in response to the capacitive sensor detecting the presence of the fluid, an alert is generated to perform a visual verification of the tubing.

4. The dialysis machine according to claim 3, wherein in response to a lack of presence of the fluid in the visual verification of the tubing, an alarm is generated by the dialysis machine.

5. The dialysis machine according to claim 3, wherein in response to a confirmation of presence of the fluid in the visual verification of the tubing, a dialysis treatment is performed.

6. The dialysis machine according to claim 1, further comprising a timer for the fluid flow through the tubing to the connector, wherein in response to exceeding a predetermined time to detect the presence of the fluid, an alarm is generated by the dialysis machine.

7. The dialysis machine according to claim 1, wherein the capacitive sensor is disposed in the dialysis machine in proximity to the connector, such that a capacitance in the sensor switches in response to detecting the presence of the fluid within a predetermined distance to the sensor.

8. A method for priming tubing of a dialysis machine, comprising:
attaching an end of the tubing to a connector coupled to a housing of the dialysis machine;
delivering fluid from a first container through the tubing to the connector; and
detecting a presence of the fluid at the end of the tubing by a capacitive sensor disposed in proximity to the connector.

9. The method according to claim 8, wherein the connector includes a hydrophobic filter.

10. The method according to claim 8, wherein in response to detecting the presence of the fluid, generating an alert for a visual verification of the tubing.

11. The method according to claim 10, wherein in response to a lack of presence of the fluid in the visual verification of the tubing, generating an alarm.

12. The method according to claim 10, wherein in response to a confirmation of presence of the fluid in the visual verification of the tubing, performing a dialysis treatment.

13. The method according to claim 8, further comprising timing the flow of fluid through the tubing to the connector, wherein in response to exceeding a predetermined time to detect the presence of the fluid, generating an alarm.

14. The method according to claim 8, wherein the capacitive sensor is disposed in the dialysis machine in proximity to the connector, such that a capacitance in the sensor switches in response to detecting the presence of fluid within a predetermined distance to the sensor.

15. A dialysis system, comprising:
a dialysis machine including a housing;
tubing extendable between the dialysis machine and a patient for fluid delivery from a container to the patient during a dialysis treatment;
a connector attachable to the housing and configured to receive at least an end of the tubing; and
a capacitive sensor disposed in proximity to the connector;
wherein, prior to the dialysis treatment, a fluid is flowable through the tubing from the container to the end of the tubing at the connector such that a presence of the fluid at the end of the tubing is detectable by the capacitive sensor.

16. The system according to claim 15, wherein the connector includes a hydrophobic filter.

17. The system according to claim 15, wherein in response to the capacitive sensor detecting the presence of the fluid, an alert is generated to perform a visual verification of the tubing.

18. The system according to claim 17, wherein in response to a lack of presence of the fluid in the visual verification of the tubing, an alarm is generated.

19. The system according to claim 17, wherein in response to a confirmation of presence of the fluid in the visual verification of the tubing, the dialysis treatment is performed.

20. The system according to claim 15, further comprising a timer for the fluid delivery through the tubing to the end of the tubing, wherein in response to exceeding a predetermined time to detect the presence of the fluid, an alarm is generated by the system.

21. The system according to claim 15, wherein the capacitive sensor is disposed in the dialysis machine in proximity to the connector, such that a capacitance in the sensor switches in response to detecting the presence of the fluid within a predetermined distance to the sensor.

22. The system according to claim 15, wherein the dialysis machine is a peritoneal dialysis machine.
